(19) United States
(12) Patent Application Publication
(10) Pub. No.: US 2025/0161396 A1
(43) Pub. Date: May 22, 2025

(54) COMPOSITION AND METHOD FOR IMPROVING LIPID METABOLISM FUNCTION OF SUBJECT

(71) Applicant: NANJING NUTRABUILDING BIO-TECH CO., LTD., Nanjing (CN)

(72) Inventors: Rui SHU, Nanjing (CN); Kylin LIAO, Nanjing (CN); Mingru WANG, Nanjing (CN)

(21) Appl. No.: 19/035,404

(22) Filed: Jan. 23, 2025

Related U.S. Application Data
(63) Continuation-in-part of application No. PCT/CN2023/096261, filed on May 25, 2023.

(30) Foreign Application Priority Data
Aug. 11, 2022 (CN) CN202210961585.8

(51) Int. Cl. A61K 36/906; A61K 31/165; A61K 31/197; A61K 31/205; A61K 35/60; A61K 45/06; A61P 3/04; A61P 3/06; A61P 3/08

(52) U.S. Cl. CPC A61K 36/906; A61K 31/165; A61K 31/197; A61K 31/205; A61K 35/60; A61K 45/06; A61P 3/04; A61P 3/06; A61P 3/08

(57) ABSTRACT

The invention discloses a method for improving lipid metabolism in a subject, which comprises administering to the subject a composition comprising: an effective amount of beta-aminoisobutyric acid or a pharmaceutically acceptable salt, acid, ester or derivative thereof; as well as an effective amount of Grains of Paradise or Aframomum melegueta. The invention also discloses the composition and use thereof in preparing a nutritional supplement, food, beverage, animal feed or medicine for improving lipid metabolism in a subject. The method and composition of the invention may reduce body weight, reduce body fat mass, improve glucose tolerance and insulin sensitivity, decrease triglyceride and total cholesterol levels, or decrease leptin level; thereby reducing obesity epidemic and obesity-related metabolic disorders worldwide and improving health status.

### CROSS REFERENCE TO RELATED APPLICATION

This application is a continuation-in-part application of International Patent Application No. PCT/CN2023/096261, filed on May 25, 2023, which claims the priority of the Chines patent application No. CN2022109615858, filed on Aug. 11, 2022, the contents of all of which are incorporated herein by reference in its entirety.

### FIELD OF THE INVENTION

The invention belongs to the technical field of health foods and dietary supplements. In particular, the invention relates to a composition and a method for improving lipid metabolism in a subject, and use thereof in preparing a nutritional supplement, food, beverage, animal feed or medicine for improving lipid metabolism in a subject.

### BACKGROUND OF THE INVENTION

With the many recent advances in the biomedical world, vast changes are taking place in our growing knowledge of the physiological aspects of almost all the tissues and organs of the human body. One of the most prevalent topics of discussion is the question of obesity and its effect on the metabolic changes in the human body. The original classical role of adipose tissue as an energy storage organ has been greatly modified. We now know that it is an endocrine organ, producing adipokines like leptin, adiponectin, visfatin, resistin, apelin, etc., which modulate metabolic processes in the body. Since obesity is associated with an increase in the adipose tissue mass, these hormones may be expected to be produced in increased concentrations and may thus have a significant impact on the macronutrient metabolism. The worldwide obesity epidemic—along with an array of obesity-related metabolic disorders, particularly diabetes, fatty liver and cardiovascular diseases—has become a major public health threat in the 21st century.

β-aminoisobutyric acid (BAIBA) is a non-protein amino acid secreted by skeletal muscles upon regular exercise via peroxisome proliferator-activated receptor gamma coactivator 1-alpha (PGC-1)-dependent mechanism. BAIBA has been discovered as a novel endogenous protective myokine regulating adipose tissue browning, changing the white adipose tissue (WAT) into brown adipose tissue (BAT). There are two enantiomers of BAIBA in biological systems: D-BAIBA and L-BAIBA. L-BAIBA is generated from catabolic reactions of branched-chain amino acid L-valine, D-BAIBA is produced in the cytosol from thymine in a metabolic pathway.

Grains of Paradise, GP (Aframomum melegueta), also known as alligator pepper or Guinea pepper, is an ancient spice from the ginger family (Zingiberaceae). GP offers powerful anti-obesity potential by activating brown adipose tissue (BAT), leading to an increase in energy expenditure.

Therefore, it is particularly important to study improved methods and compositions to improve health by improving lipid metabolism.

### SUMMARY OF THE INVENTION

BAIBA “prepares” the fuels ready—the BAT, then GP “ignites” these fuels. The combination of the two can significantly improve lipid metabolism, reduce body weight, reduce body fat mass and adjust relevant biochemical indexes, such as improving glucose tolerance and insulin sensitivity, decreasing triglyceride and total cholesterol levels, decreasing leptin level, etc., thereby improving health status and/or bringing better weight loss effect, so as to better improve health status.

To achieve the above object, in one aspect, the present invention provides a method for improving lipid metabolism in a subject, which comprises administering to the subject a composition comprising: an effective amount of beta-aminoisobutyric acid (BAIBA) or a pharmaceutically acceptable salt, acid, ester or derivative thereof as a first component; and an effective amount of Grains of Paradise (GP) or Aframomum melegueta as a second component. In some embodiments, the beta-aminoisobutyric acid (BAIBA) in the composition includes L-BAIBA and D-BAIBA. In some embodiments, the ratio of L-BAIBA and D-BAIBA is not 1:1. In some embodiments, it may be necessary to provide a high content of L-BAIBA and a low content of D-BAIBA in the composition, while in other embodiments, it may be necessary to provide a high content of D-BAIBA and a low content of L-BAIBA. In some embodiments, the content of L-BAIBA in the composition is higher than that of D-BAIBA. In some embodiments, 55% of BAIBA in the composition is L-BAIBA, and 45% of BAIBA is D-BAIBA.

In some embodiments, the method of the invention is used for improving lipid metabolism in a subject. In some embodiments, the method of the invention is also used for reducing body weight, reducing body fat mass, improving glucose tolerance and insulin sensitivity, decreasing triglyceride and total cholesterol levels, or decreasing leptin level in the subject. In other embodiments, the method of the invention can realize one or more of the above or any combination thereof.

In some preferred embodiments, the administered composition can be formulated as a nutritional supplement, food, beverage, animal feed or medicine, etc. In other preferred embodiments, the administered composition is in the form of suppository, tablet, pill, granule, powder, film, capsule, beverage, aerosol, elixir, tincture, tonic, liquid suspension or syrup, etc.

In some preferred embodiments, the weight ratio of the first component to the second component is 1.5 to 40:1. An appropriate weight ratio can be selected according to requirements, preferably 3-20:1, 6-30:1, 7-25:1, 8-20:1; more preferably 4-15:1, 7-15:1, 9-15:1; and most preferably 10-15:1, 12.5:1. In other preferred embodiments, the first component is administered in an amount of 50-5000 mg/day, preferably 60-3500 mg/day, more preferably 100-2500 mg/day, and most preferably 500-1500 mg/day; the second component is administered in an amount of 4-400 mg/day, preferably 5-300 mg/day, more preferably 10-260 mg/day, most preferably 40-160 mg/day.

In some preferred embodiments, the method of the invention reduces the body weight of the subject by 10%, 15%, 20%, 25%, 30%, 35%, 40%. In some preferred embodiments, the method of the invention reduces the body weight of the subject to be close to normal level, and/or restores it to be healthy level.

In some preferred embodiments, the method of the invention reduces the body fat mass of the subject by 15%, 20%, 25%, 30%, 35%, 40%, 45%. In some preferred embodiments, the method of the invention decreases the triglyceride (TG) level in the subject by 25%, 30%, 35%, 40%, 45%, 50%, 55%. In some preferred embodiments, the method of the invention decreases the total cholesterol (TC) level in the subject by 15%, 20%, 25%, 30%.

In some preferred embodiments, the method of the invention decreases the leptin level in the subject by 20%, 25%, 30%, 35%. In some preferred embodiments, the method of the invention decreases the leptin level in the subject to be close to normal level, and/or restores it to be healthy level.

In another aspect, the present invention provides a composition comprising beta-aminoisobutyric acid (BAIBA) or a pharmaceutically acceptable salt, acid, ester or derivative thereof as a first component; and Grains of Paradise (GP) or Aframomum melegueta as a second component. In some embodiments, the ratio of L-BAIBA and D-BAIBA is not 1:1. In some embodiments, it may be necessary to provide a high content of L-BAIBA and a low content of D-BAIBA in the composition, while in other embodiments, it may be necessary to provide a high content of D-BAIBA and a low content of L-BAIBA. In some embodiments, the content of L-BAIBA in the composition is higher than that of D-BAIBA. In some embodiments, 55% of BAIBA in the composition is L-BAIBA, and 45% of BAIBA is D-BAIBA. In some embodiments, the weight ratio of L-BAIBA to D-BAIBA in the composition is no less than 55/45, 53/47, or 51/49, or the weight ratio of D-BAIBA to L-BAIBA in the composition is no less than 55/45, 53/47, or 51/49. In some embodiments, the BAIBA is at least about 51%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or substantially all L-β-aminoisobutyric acid (L-BAIBA). In some embodiments, the composition of the invention can also contain other similar substances that can transform white adipose tissue (WAT) into brown adipose tissue (BAT), such as resveratrol, curcumin, menthol, ursolic acid, tea polyphenols, coenzyme Q10, caffeine, etc.; and other effective brown adipose activators, such as capsaicinoid, and any other substances that can produce similar effects to GP, such as L-carnitine, capsaicin, choline, fish oil, etc.

In some embodiments, the composition of the invention is used for improving lipid metabolism in a subject. In some embodiments, the composition of the invention is used for reducing body weight, reducing body fat mass, improving glucose tolerance and insulin sensitivity, decreasing triglyceride and total cholesterol levels, or decreasing leptin level in the subject, or any combination thereof.

In some preferred embodiments, the composition of the invention can be formulated as a nutritional supplement, food, beverage, animal feed or medicine, etc. In other preferred embodiments, the composition of the invention is in the form of suppository, tablet, pill, granule, powder, film, capsule, beverage, aerosol, elixir, tincture, tonic, liquid suspension or syrup, etc.

In some preferred embodiments, the weight ratio of the first component to the second component in the composition of the invention is 1.5 to 40:1; an appropriate weight ratio can be selected according to requirements, preferably 3-20:1, 6-30:1, 7-25:1, 8-20:1; more preferably 4-15:1, 7-15:1, 9-15:1; and most preferably 10-15:1, 12.5:1. In other preferred embodiments, the first component is administered in an amount of 50-5000 mg/day, preferably 60-3500 mg/day, more preferably 100-2500 mg/day, most preferably 500-1500 mg/day; the second component is administered in an amount of 4-400 mg/day, preferably 5-300 mg/day, more preferably 10-260 mg/day, most preferably 40-160 mg/day.

In another aspect, the present invention provides use of a composition in preparing a nutritional supplement, food, beverage, animal feed or medicine for improving lipid metabolism in a subject, the composition comprises beta-aminoisobutyric acid (BAIBA) or a pharmaceutically acceptable salt, acid, ester or derivative thereof as a first component; and Grains of Paradise (GP) or Aframomum melegueta as a second component.

In some embodiments, the composition can improve lipid metabolism in a subject. In some embodiments, the composition is used for reducing body weight, reducing body fat mass, improving glucose tolerance and insulin sensitivity, decreasing triglyceride and total cholesterol levels, or decreasing leptin level in the subject, or any combination thereof.

In some preferred embodiments, the composition is administered in the form of a suppository, tablet, pill, granule, powder, film, capsule, beverage, aerosol, elixir, tincture, tonic, liquid suspension or syrup, etc.

In some preferred embodiments, the composition is administered to the subject in an amount of 50-5000 mg of the first component and 4-400 mg of the second component per day. The dosage of the first component is preferably 60-3500 mg, more preferably 100-2500 mg, and most preferably 500-1500 mg per day; the dosage of the second component is preferably 4-400 mg, preferably 5-300 mg, more preferably 10-260 mg, and most preferably 40-160 mg per day. In some preferred embodiments, the weight ratio of the first component to the second component is 1.5 to 40:1; An appropriate weight ratio can be selected according to requirements, preferably 3-20:1, more preferably 4-15:1, and most preferably 12.5:1.

In some preferred embodiments, the nutritional supplement, food, beverage, animal feed or medicine also comprises a dietetically or pharmaceutically acceptable carrier, including a liquid or solid filler, a diluent, an excipient, a solvent or an encapsulating material.

The beneficial effects of the method and composition of the present invention include improving lipid metabolism in a subject, and/or reducing body weight, reducing body fat mass, improving glucose tolerance and insulin sensitivity, and reducing triglyceride and total cholesterol levels and leptin levels; and can be used in various applications such as nutritional supplements, foods, beverages, animal feeds or medicines, etc., thereby reducing obesity epidemic and obesity-related metabolic disorders worldwide and improving health status. In addition, when administered to a subject, the composition shows better comprehensive effects than the individual components, and the desired effects can be achieved with less dosage, and the adaptability and tolerance are superior.

### BRIEF DESCRIPTION OF DRAWINGS

FIG. 1 is a comparison diagram of the body weights of five groups of mice without or with different substances.

FIG. 2 is a comparison diagram of body fat masses of five groups of mice without or with different substances.

FIGS. 3A and 3B are graphs of blood glucose changes of five groups of mice without administration, or before or 15, 30, 60, 90 and 120 minutes after administration of different substances in OGTT and ITT experiments, respectively.

FIGS. 4A and 4B are the comparison diagrams of triglyceride (TG) and total cholesterol (TC) levels in five groups of mice without or with administration of different substances, respectively.

FIG. 5 is a comparison diagram of leptin levels in five groups of mice without or with administration of different substances.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

The terms ‘comprising’ or ‘to comprise’ and their conjugations, as used herein, refer to a situation wherein said terms are used in their non-limiting sense to mean that items following the word are included, but items not specifically mentioned are not excluded. It also encompasses the more limiting verb ‘to consist essentially of’ and ‘to consist of’.

As used herein, the terms “subject” or “individual” are used interchangeably to refer to any subject to whom the disclosed methods and compositions can be applied or administered. The subject may have a disease or disorder, but the subject does not need to be sick to benefit from the disclosed methods and compositions. Subjects may need to improve their lipid metabolism and/or overall health, but they may also have overall healthy lipid metabolism and other metabolism and wish to maintain or further improve their lipid metabolism and/or overall health. Therefore, any subject can take the disclosed composition or become a recipient of the disclosed method. As used herein, the term “subject” refers to animals (for example, birds, reptiles and mammals). In some embodiments, the subject can be mammals including non-primates (e.g., camels, donkeys, zebras, cows, horses, cats, dogs, rats and mice) and primates (e.g., monkeys, chimpanzees and humans). In certain embodiments, the subject may be a non-human mammal. In other embodiments, the subject may be a human.

As used herein, the term “administration” refers to the process of delivering the disclosed composition to a subject. The composition can be administered in various ways, including oral, intragastric and parenteral (i.e. intravenous and intra-arterial and other suitable parenteral routes), etc.

As used herein, the term “effective amount” refers to the amount required to achieve an effect as taught herein. For example, the amount required to improve lipid metabolism in a subject, such as or including (but not limited to) reducing body weight, reducing body fat mass, improving glucose tolerance and insulin sensitivity, decreasing triglyceride and total cholesterol levels, and decreasing leptin levels. According to the present disclosure, a suitable single dose size is a dose that can improve the lipid metabolism of a subject when administered once or more times within a suitable period of time.

As used herein, the term “dietetically or pharmaceutically acceptable carrier” is used interchangeably with “physiologically acceptable carrier” or “nutritionally acceptable carrier”, and the administration form of the composition of the present invention involves liquid or solid fillers, diluents, excipients, solvents or encapsulating materials. Each carrier must be “acceptable”, which means that it is compatible with other components of the composition and harmless to the subject, that is, suitable for consumption or nutritionally acceptable. The above carriers include non-toxic and compatible substances commonly used in health foods, dietary supplements and pharmaceutical preparations, such as sugar, starch, cellulose and its derivatives, powdered tragacanth, malt, gelatin, talc, oil, diol, polyol, ester, agar, alginic acid, pyrogen-free water, isotonic saline, etc.

The method of the present invention comprises the daily administration of at least 50 mg of BAIBA or a pharmaceutically acceptable salt, acid, ester or derivative thereof and at least 4 mg of GP, depending on the specific preparation and form containing two components. In some embodiments, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or substantially all of the BAIBA is L-BAIBA. In some embodiments, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or substantially all of the BAIBA is D-BAIBA. The amount to be administered can also vary according to factors such as sensitivity, age, sex and weight of the subject, specific reaction, etc. One or more doses can be administered once or more times a day or at a suitable frequency for any period of time. For example, an effective dose can be administered daily for one day, several days, many days or indefinitely.

In some embodiments, the composition of the present invention can be administered together with other supplements, such as vitamins, minerals, tonics and any other suitable supplements known in the art.

The composition of the present invention can be prepared into nutritional supplements or dietary supplements, (medical) foods, animal feeds or pharmaceutical compositions in liquid or solid form using standard excipients and preparation techniques. For example, where the composition is in solid form, the compositions may be formulated as a snack bar, yogurt, lozenge, tablet, or capsule, or may be coated onto cereal products, included in baked goods. On the other hand, where the supplement is in liquid form, the compositions may be formulated as a tincture, soft gel capsule, liquid capsule, syrup, carbonated drink, a brewed beverage (e.g., as coffee or tea), a juice, an energy drink, a sports drink, or flavored water. While nutritional and pharmaceutical compositions for human use are especially contemplated, it should be appreciated that the compositions and formulations of the invention may also be employed for veterinary use (e.g., use in animal feed for domestic companion animals (‘pets’) or in animal feed for farm animals. In further contemplated aspects, the composition of the invention may also be provided as a bulk product (e.g., in quantities of equal or greater than 100 g, equal or greater than 1,000 g, or equal or greater than 10 kg) for use in production of the nutritional supplement, a (medical) food item, animal feed, or pharmaceutical product.

The composition of the present invention can be administered to a subject in any suitable way, including (but not limited to) oral administration in a solid dosage form or through parenteral or transdermal administration to exert the desired effect.

### EXAMPLES

Mice were divided into five groups: one group was normal diet mice as ND control group; one group of high fat diet (HFD) mice were gavaged with no-active-ingredients water as HFD control group, one group of HFD mice were treated with BAIBA (65 mg/kg/day) alone by gavage as HFD+BAIBA group, one group of HFD mice were treated with GP (5.2 mg/kg/day) alone by gavage as HFD+GP group, and one group of HFD mice were treated with BAIBA (65 mg/kg/day) and GP (5.2 mg/kg/day) combination by gavage as HFD+BAIBA+GP group, all for 16 weeks.

### Example 1 the Combination of BAIBA and GP Reduces Body Weight

At the beginning of treatment (baseline), the body weights of the five groups of mice were the same. After treatment (16 weeks), as shown in FIG. 1, the body weights of mice in ND control group, HFD control group, HFD+BAIBA group, HFD+GP group and HFD+BAIBA+GP group were 28.4 g, 49.2 g, 47.8 g, 46.2 g and 33.6 g respectively. The weight of HFD mice treated with BAIBA alone and GP alone reduced by 2.84% and 6.09% respectively; while the weight of HFD mice treated with BAIBA+GP reduced by 31.71%, which is the closest to that of normal mice. It can be seen that the combination of BAIBA and GP can significantly reduce body weight, and the effect is obviously better than the simple accumulation when they are used alone.

### Example 2 the Combination of BAIBA and GP Reduces Body Fat Mass

Adipose tissue was collected, fixed with 10% formalin, embedded in paraffin, and dehydrated by graded alcohol. Tissue sections with a thickness of 5 μm were prepared, stained with hematoxylin-eosin (H&E) and observed under an optical microscope with a magnification of 200 times. The body fat mass of each group of mice was measured and recorded.

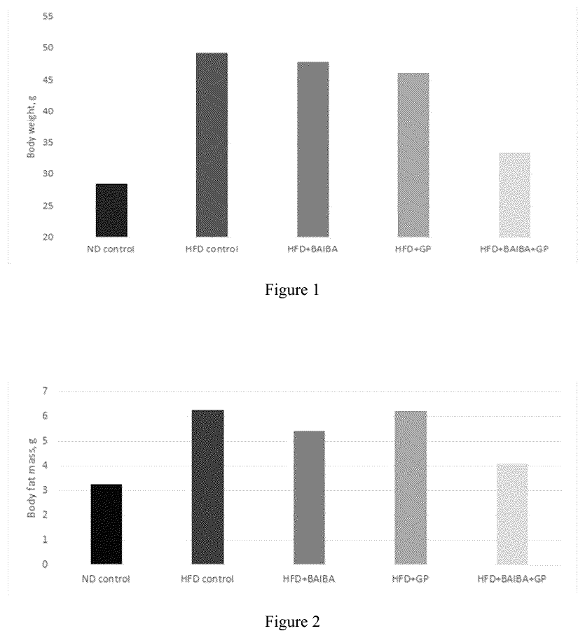

After treatment (16 weeks), as shown in FIG. 2, the body fat masses of mice in ND control group, HFD control group, HFD+BAIBA group, HFD+GP group and HFD+BAIBA+GP group were 3.25 g, 6.24 g, 5.38 g, 6.21 g and 4.1 g, respectively. The body fat mass of HFD mice treated with BAIBA alone reduced by 13.78%, that of HFD mice treated with GP alone remained almost unchanged; while the body fat mass of HFD mice treated with BAIBA+GP reduced by 34.29%. It can be seen that the combination of BAIBA and GP can significantly reduce body fat mass, and the effect is obviously better than the simple accumulation when they are used alone.

### Example 3 the Combination of BAIBA and GP Improves Glucose Tolerance and Insulin Sensitivity

At the 8th week after the initial experiment, oral glucose tolerance test (OGTT) and insulin tolerance test (ITT) were performed on mice. OGTT is as follows: Blood from tail tip of mice was taken to detect blood glucose, and glucose (1.5 g/kg) was given by gavage after overnight fasting, and blood glucose was monitored with a blood glucose meter before administration and 15, 30, 60, 90 and 120 minutes after administration. ITT is as follows: Insulin (0.75 U/kg) was given by intraperitoneal injection on an empty stomach, and blood glucose was monitored with a blood glucose meter before administration and 15, 30, 60, 90 and 120 minutes after administration.

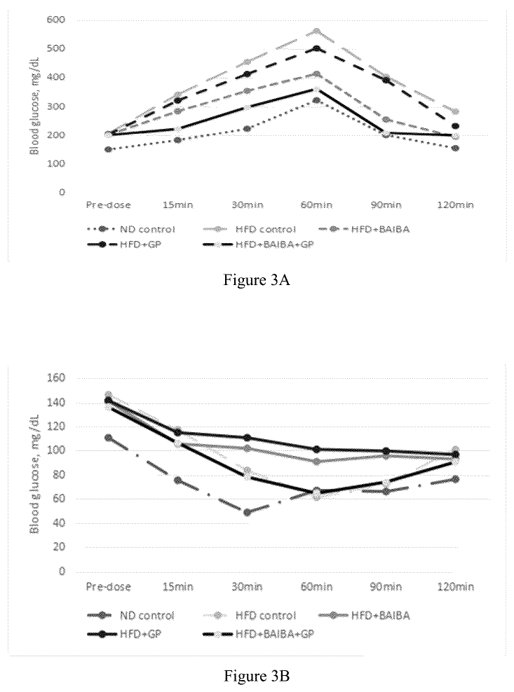

Table 1 shows the blood glucose levels (mg/dL) of mice at various time points in OGTT; the change curve is shown in FIG. 3A. As shown in Table 1 and FIG. 3A, after 15 minutes, the blood glucose level of mice in HFD control group increased rapidly with a large range, but decreased relatively slowly, indicating that the mice in this group had impaired glucose tolerance; the blood glucose level of mice in HFD+BAIBA group was lower than that in HFD control group at each time point, and the upward and downward trends were gentle; the blood glucose level of mice in HFD+GP group was lower than that in HFD control group and higher than that in HFD+BAIBA group; the blood glucose level of mice in HFD+BAIBA+GP group was significantly lower than those in other groups except ND control group at different time points, and the trend was more gentle, indicating that the mice in this group had strong tolerance to glucose. It can be seen that the combination of BAIBA and GP can significantly improve the tolerance of mice to glucose, that is, improve the glucose tolerance.

TABLE 1

| | Pre- | 15 | 30 | 60 | 90 | 120 |
|---|---|---|---|---|---|---|
| | dose | min | min | min | min | min |
| ND control | 148.57 | 180.64 | 221.33 | 320.15 | 198.24 | 152.84 |
| HFD control | 205.45 | 340.83 | 453.49 | 560.78 | 401.89 | 280.45 |
| HFD + BAIBA | 199.85 | 281.91 | 352.74 | 411.98 | 253.21 | 192.99 |
| HFD + GP | 200.94 | 318.87 | 410.93 | 500.05 | 389.48 | 230.56 |
| HFD + | 198.48 | 220.59 | 295.78 | 359.48 | 206.45 | 197.54 |
| BAIBA + GP | | | | | | |

Table 2 shows the blood glucose levels (mg/dL) of mice at various time points in ITT; the change curve is shown in FIG. 3B. As shown in Table 2 and FIG. 3B, after 15 minutes, the blood glucose level of mice in HFD control group decreased greatly, and the blood glucose level before and after the end of insulin half-life was higher than that in other groups. The blood glucose level of mice in HFD+BAIBA group was higher than that in HFD control group at each time point, and the upward and downward trends were gentle; the blood glucose level of mice in HFD+GP group was higher than that in HFD+BAIBA group; the blood glucose levels of mice in HFD+BAIBA+GP group at different time points were significantly lower than those in other groups except ND control group, indicating that there was no insulin resistance in this group. It can be seen that the combination of BAIBA and GP can reduce the insulin resistance of mice, that is, improve the insulin sensitivity.

TABLE 2

| | Pre- | 15 | 30 | 60 | 90 | 120 |
|---|---|---|---|---|---|---|
| | dose | min | min | min | min | min |
| ND control | 110.25 | 74.98 | 48.25 | 66.64 | 65.67 | 75.85 |
| HFD control | 145.97 | 116.78 | 83.25 | 61.05 | 72.16 | 100.56 |
| HFD + BAIBA | 140.4 | 105.23 | 101.45 | 90.45 | 95.24 | 92.56 |
| HFD + GP | 141.02 | 114.45 | 110.2 | 100.56 | 99.23 | 96.25 |
| HFD + | 135.54 | 105.88 | 77.66 | 64.28 | 73.56 | 90.15 |
| BAIBA + GP | | | | | | |

### Example 4 the Combination of BAIBA and GP Decreases Triglyceride (TG) and Total Cholesterol (TC) Levels

After the mice were killed, the serum was extracted by centrifugation, and the levels of triglyceride (TG) and total cholesterol (TC) in mice were measured by using commercial kits according to the instructions and recorded.

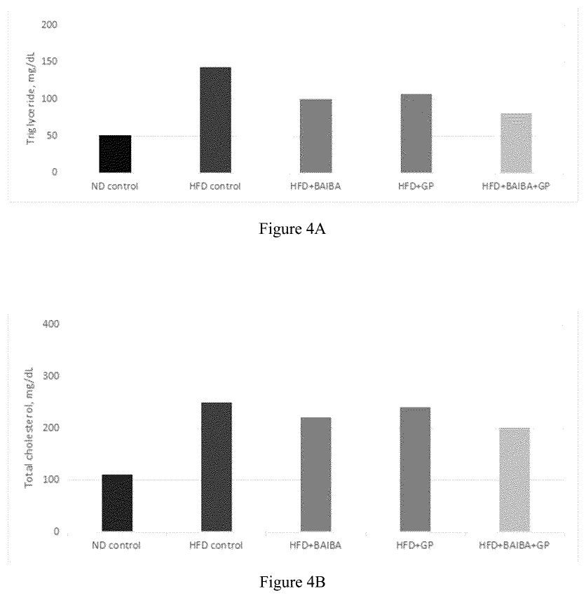

After treatment (16 weeks), as shown in FIG. 4A, the triglyceride (TG) levels of mice in ND control group, HFD control group, HFD+BAIBA group, HFD+GP group and HFD+BAIBA+GP group were 51.26 mg/dL, 142.52 mg/dL, 98.45 mg/dL, 106.21 mg/dL and 81.45 mg/dL, respectively. After the administration of BAIBA+GP, the triglyceride (TG) level of HFD mice decreased by 42.85%, which shows that the combination of BAIBA and GP can significantly reduce the triglyceride (TG) level.

After treatment (16 weeks), as shown in FIG. 4B, the total cholesterol (TC) levels of mice in ND control group, HFD control group, HFD+BAIBA group, HFD+GP group and HFD+BAIBA+GP group were 110.69 mg/dL, 249.55 mg/dL, 220.65 mg/dL, 240.69 mg/dL and 200.66 mg/dL, respectively. After the administration of BAIBA+GP, the total cholesterol (TC) level of HFD mice decreased by 19.59%, which shows that the combination of BAIBA and GP can significantly reduce the total cholesterol (TC) level.

### Example 5 the Combination of BAIBA and GP Decreases Leptin Level

The serum of mice was centrifuged, and the level of leptin in serum was determined by ELISA kit. According to the concentration value and light absorption value of leptin, the logarithmic value was taken as a standard curve, and the concentration value of leptin was converted according to the standard curve.

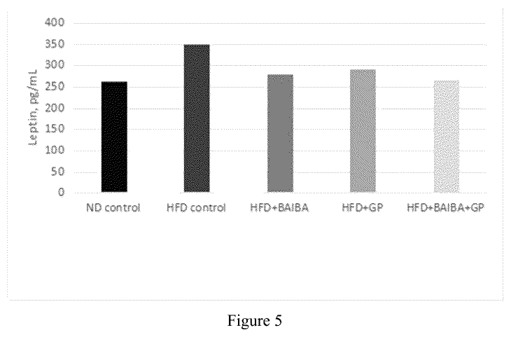

After treatment (16 weeks), as shown in FIG. 5, the leptin level of mice in ND control group, HFD control group, HFD+BAIBA group, HFD+GP group and HFD+BAIBA+GP group were 261.58 pg/mL, 349.39 pg/mL, 278.12 pg/mL, 290.85 pg/mL and 265.48 pg/mL, respectively. After the administration of BAIBA+GP, the leptin level of HFD mice decreased by 24.02%, which was close to that of normal mice. It can be seen that the combination of BAIBA and GP significantly reduced the leptin level.

The above is only the preferred embodiments of the present invention, and it is not intended to limit the present invention. Any person skilled in the art can make many changes, modifications, substitutions and variations on these embodiments without departing from the principles and purposes of the invention, and the scope of the invention is defined by the claims and their equivalents.

What is claimed is:

1. A method for improving lipid metabolism in a subject, wherein the method comprises administering to the subject a composition comprising: an effective amount of beta-aminoisobutyric acid or a pharmaceutically acceptable salt, acid, ester or derivative thereof as a first component; and an effective amount of Grains of Paradise or Aframomum melegueta as a second component.

2. The method of claim 1, wherein the method is used for reducing body weight, reducing body fat mass, improving glucose tolerance and insulin sensitivity, decreasing triglyceride and total cholesterol levels, or decreasing leptin level in the subject.

3. The method of claim 1, wherein the composition is formulated as a nutritional supplement, food, beverage, animal feed.

4. The method of claim 1, wherein the weight ratio of the first component to the second component is 1.5 to 40:1.

5. The method of claim 1, wherein the first component is administered in an amount of 50-5000 mg/day, the second component is administered in an amount of 4-400 mg/day.

6. The method of claim 1, wherein the ratio of L-BAIBA and D-BAIBA in the composition is not 1:1.

7. The method of claim 1, wherein the weight ratio of L-BAIBA to D-BAIBA in the composition is no less than 55/45, or the weight ratio of D-BAIBA to L-BAIBA in the composition is no less than 55/45.

8. The method of claim 1, wherein the composition comprises other substances that can transform white adipose tissue into brown adipose tissue, including resveratrol, curcumin, menthol, ursolic acid, tea polyphenols, coenzyme Q10, caffeine.

9. The method of claim 1, wherein the composition comprises L-carnitine, capsaicin, choline, or fish oil.

10. The method of claim 1, wherein the composition is in the form of suppository, tablet, pill, granule, powder, film, capsule, beverage, aerosol, elixir, tincture, tonic, liquid suspension or syrup.

11. The method of claim 1, wherein the weight ratio of the first component to the second component is 8 to 20:1.
